(19) United States
(12) Patent Application Publication
(10) Pub. No.: US 2011/0244060 A1
(43) Pub. Date: Oct. 6, 2011

(54) CARDIOPROTECTIVE EFFECTS OF NUTRACEUTICALS ISOLATED FROM NIGELLA SATIVA SEEDS

(75) Inventors: Maznah Ismail, Selangor Darul Ehsan (MY); Ghanya Al-Naqeeb, Selangor Darul Ehsan (MY)

(73) Assignee: UNIVERSITI PUTRA MALAYSIA, UPM Serdang, Selangor Darul Ehsan (MY)

(21) Appl. No.: 13/124,370

(22) PCT Filed: Aug. 7, 2009
(86) PCT No.: PCT/MY09/00114

§ 371 Date: May 26, 2011

(30) Foreign Application Priority Data
Oct. 28, 2008 (MY) PI 20084285

(51) Int. Cl. A61K 36/71; C07C 50/02; A61P 9/00; A61P 3/06; A61P 39/06

(52) U.S. Cl. 424/725; 552/309

(57) ABSTRACT

This invention relates to a rich antioxidant nutraceuticals prepared from Nigella sativa seeds for the treatment and prevention of hypercholesterolemia. These nutraceuticals when taken enterally has shown successfully to have hypocholesterolmic, antiatherogenic and antioxidant activities using in vitro and in vivo study. The invention provides a clear explanation on the molecular mechanism by which nutraceuticals prepared from the Nigella sativa seeds exerted its hypocholesterolemic effect.

### FIELD OF INVENTION

The present invention relates to cardioprotective effects of nutraceuticals isolated from Nigella sativa seeds.

### BACKGROUND OF INVENTION

Cardiovascular diseases (CVD), including coronary heart disease (CHD) and atherosclerosis are the common causes of death worldwide (Wallidus et al., 1993). The risk of CHD is closely related with elevated plasma levels of total cholesterol (TC), low density lipoprotein cholesterol, (LDLC) as well as low level of high density lipoprotein cholesterol (HDLC) (Hironori et al., 2005).

While cholesterol is a major component of cell membranes, it is essential for tissue growth, bile acids, lipoproteins, and steroid hormone (Tabas, 2002). However, excess of cholesterol can be toxic to cell functions and may disrupt the plasma membrane. Therefore, its level needs to be maintained within normal range to preserve normal cell functions. In humans, maintaining cholesterol level is dependent on coordinated changes in the levels of mRNAs of key genes that are known to regulate cholesterol synthesis and cholesterol uptake from plasma (Horton et al., 2003). Many genes including low-density lipoprotein receptor (LDLR), 3-hydroxy-3-methylglutaryl CoA reductase (HMG-COAR) apolipoprotein (Apo) B100, Apo E and Apo A-1 genes have been identified and found to regulate cholesterol metabolism.

Statins are the current pharmacological drug, which used as lipid lowering agent in the clinical practice nowadays. Side effects such as hepatotoxicity and myotoxicity have been closely associated with statins (Masters, 1995). Given the high rate of side effects, significant emphasis has been recently placed on the use of statins, especially with natural occurring products. Furthermore, most of the cholesterol-lowering compounds that reviewed in the literature required further studies to confirm the findings, the safety of selected dosage and understanding the mechanism of action. In this invention, nutraceuticals prepared from Nigella sativa seeds hold promise that can prevent and treat hypercholesterolemia.

Nigella sativa (Black seed) belongs to Ranunculaceae family contain more than 30% of fixed oil and 0.40% to 0.45% of volatile oil (Ali and Blunden, 2003). Thymoquonine (TQ) is the major bioactive component (18.4% to 24%) in Nigella sativa volatile oil (Arslan et al., 2005). The seeds have been most extensively both pharmacologically and phytochemically studied in recent years for its medicinal uses (Ghosheh et al., 1998). Some of these therapeutic effects have been documented including, anti-inflammatory (Houghton et al., 1995), anti-tumour and (El-Daly, 1998) anti-diabetic (Kanter et al., 2003). Thymoquinone (TQ) has been reported to have potent superoxide anion (O₂⁻) that can inhibit the lipid peroxidation (Badary et al., 2003). Despite this knowledge about the potent, cholesterol lowering, anti-oxidant properties of TQ, and Nigella sativa oil the molecular pathways involved in these activities have not yet been reported in the literature.

The hypocholesterolemic and antioxidant effects of Nigella sativa seed powder (NSP), Nigella sativa oil (NSO), thymoquinone rich fraction (TQRF) and thymoquinone (TQ) were investigated in vitro using human HepG2 cells and in vivo using animal models (rabbits and rats). TQRF was prepared using super critical fluid extraction (SFE). This invention also provided new findings about the anti-atherogenic effect of NSO and NSP in vivo using rabbits. To the best of our knowledge, no study has been reported about the TQRF, and this is the only study which showed clear understanding of the underlying molecular mechanisms by which TQRF and TQ exert their hypocholesterolemic effects. This invention also provided new experimental data using Electron Spin Resonance (ESR) technique showing the antioxidant activity of both TQRF and TQ as scavenger for OH radical in vivo and thereby reducing serum LDL cholesterol levels.

### SUMMARY OF THE INVENTION

Accordingly, the present invention provides a use of an effective amount of thymoquinone rich fraction (TQRF) obtained from Nigella sativa using super critical fluid extraction (SFE) in the manufacture of a medicament for preventing and treating cardiovascular related diseases in a patient in need thereof.

Further, the present invention also provides a use of an effective amount of Nigella sativa powder obtained from Nigella sativa in the manufacture of a medicament for preventing and treating cardiovascular related diseases in a patient in need thereof.

Also provided, the present invention relates to a use of an effective amount of Nigella sativa oil obtained from Nigella sativa in the manufacture of a medicament for preventing and treating cardiovascular related diseases in a patient in need thereof.

Also provided, the present invention relates to a use of an effective amount of Thymoquinone (TQ) emulsion extracted from Nigella sativa oil obtained from Nigella sativa in the manufacture of a medicament for preventing and treating cardiovascular related diseases in a patient in need thereof.

### BRIEF DESCRIPTION OF THE DRAWINGS

The embodiment of the invention will now be described, by way of example only, with tables and figure in which:

FIG. 1 shows the changes of TC level through the experiment time. Results are expressed as means±SDV of 7 rats per group. PC=cholesterol positive control, NC=negative control, TQRF 1=group that treated with TQRF at 0.5 g/kg for 8 weeks, TQRF 2=group that treated with TQRF at dose 1 g/kg for 8 weeks, TQRF 3=group that treated with TQRF at dose of 1.5 g/kg for 8 weeks. TQ 1=group that treated with TQ at dose 20 mg/kg for 8 weeks, TQ 2=group that treated with TQ at dose of 50 mg/kg for 8 weeks, TQ 3=group that treated with TQ at dose 20 mg/kg for 8 weeks.

FIG. 2 shows changes of total cholesterol in hypercholesterolemia induced rabbits. Results are expressed as means±SD of five rabbits per group. Within a column, values with the same superscript letters are not significantly different from each other. PC=positive control, NC=negative control, NSO=Nigella sativa seed oil, NSP=Nigella sativa seeds in powder form and ST=simvastatin. P<0.05 comparison of total plasma cholesterol (mmol/l) values at various times.

FIG. 3 shows hydroxyl radical scavenging activity of TQ and TQRF in plasma samples collected from experimental rats after 8 weeks of treatments during the reaction of FeSO₄ (0.2 mM), H₂O₂ (1 mM), and 40 μL 5,5-dimethyl-1-pyrroline-N-oxide (DMPO) 112.5 μL of 0.2 mM EDTA. PC=cholesterol positive control, NC=negative control, TQRF 1=group that treated with TQRF at 0.5 g/kg for 8 weeks, TQRF 2=group that treated with TQRF at dose 1 g/kg for 8 weeks, TQRF 3=group that treated with TQRF at dose of 1.5 g/kg for 8 weeks. TQ 1=group that treated with TQ at dose 20 mg/kg for 8 weeks, TQ 2=group that treated with TQ at dose of 50 mg/kg for 8 weeks, TQ 3=group that treated with TQ at dose 20 mg/kg for 8 weeks.

FIGS. 4 (a) to (e) shows representative hematoxylin and eosin stained of the intimal thickening of aorta using an image-analysis system interfaced to a Zeiss Axioscop microscope (×10). I=intima; M=media; A=adventitia; L=lumen; NC=negative control group; PC=positive control. NSP=Nigella sativa in powder form; NSO=Nigella sativa oil, ST=simvastatin group (×10);

FIG. 5 shows effect of Nigella sativa and simvastatin treatments on the intima and media thickness of aorta. The cross-sections of thoracic aorta were stained with hematoxylin-eosin. PC=positive control, NSO=Nigella sativa seed oil, NSP=Nigella sativa seeds in powder form and ST=simvastatin. Values are means±SD (n=5). Values with the same superscript letters are not significantly different from each other (p<0.0).

FIG. 6 shows effect of TQRF and TQ treatment on LDLR mRNA levels in HepG2 cells with or without 25OH. Cells were incubated for 24 hours in 10% HLPDS, and treated with 80, 50 μg/ml of TQRF and 2 μg/ml of TQ in present or absence of 2 μg/mL 25OH. mRNA level of LDLR expression was measured using quantitative real-time RT-PCR and normalized by the quantity of beta-actin mRNA. cDNAs were analyzed in trireplicate. Data represent the mean±SD. within each group results are significantly different from the control at p<0.05 level of significant) (n=3).

FIG. 7 shows regulation of LDLR gene by TQRF and TQ treatments in experimental rats. Expression mRNA level of LDLR expression was measured using quantitative real-time RT-PCR and normalized by the quantity of beta-actin mRNA. Gene expression was measured in 4 animals for each group. cDNAs were analyzed in trireplicate. Data represent the mean±SD.

FIG. 8 shows ALT level of experimental rats. Results are expressed as means±SDV of 7 rats per group. PC=cholesterol positive control, NC=negative control, TQRF 1=group that treated with TQRF at 0.5 g/kg for 8 weeks, TQRF 2=group that treated with TQRF at dose 1 g/kg for 8 weeks, TQRF 3=group that treated with TQRF at dose of 1.5 g/kg for 8 weeks. TQ 1=group that treated with TQ at dose 20 mg/kg for 8 weeks, TQ 2=group that treated with TQ at dose of 50 mg/kg for 8 weeks, TQ 3=group that treated with TQ at dose 20 mg/kg for 8 weeks.

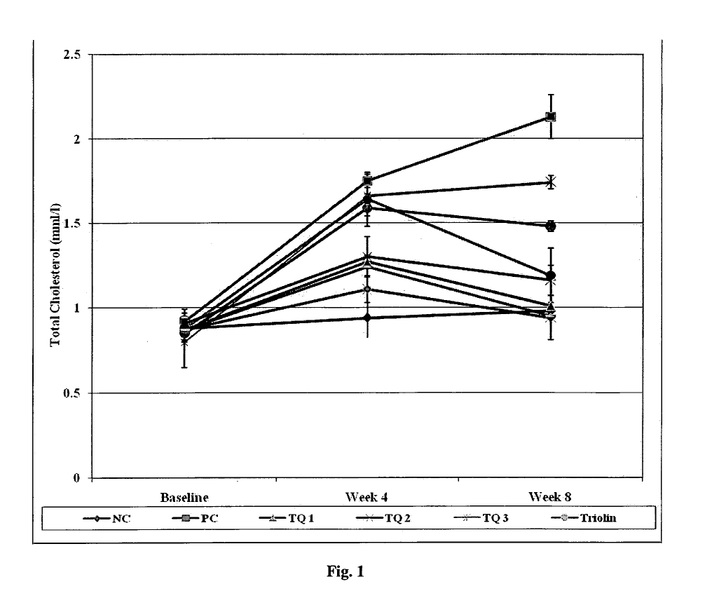

### DETAILED DESCRIPTION OF THE INVENTION

Nigella sativa L which belongs to Ranuculaceae family has been known for thousands of years as a spice and food preservative, as well as protective and curative agents for several diseases. The present invention provides SFE extraction procedure to prepare TQRF from Nigella sativa seeds. The present invention also provides a method for treating hypercholesterolemia and atherosclerosis in mammals in which rabbits and rats were used.

Male New Zealand white rabbits were firstly induced with hypercholesterolemia by feeding rabbit's diet supplemented with 0.5% cholesterol, for 3 weeks and then fed with NSP at doses ranged between 1-3.5 g/kg body weight of NSP and NSO at doses ranged between 0.5-1.5 g/kg of mixed with diet for 8 weeks. In the present invention, the in vivo (rabbit study) antioxidant activity of NSP and NSO were measured using Selectra E manufacture Vital scientific machine (UK) and with commercial kits (Randox, Crumlin, Co. Antrim, UK). The present invention provides a method of the antioxidant activity of TQRF and TQ on .OH scavenging activity using ESR in experimental rats. The present invention provided clear understanding of the molecular mechanism by which TQRF and TQ exert their lowering cholesterol property. The regulatory effect of TQRF and TQ on genes involved in cholesterol metabolism such as LDLR, HMG-COAR, Apo A-1, Apo B100 and Apo E were investigated in vitro using HepG2 cells and in vivo using Sprague-Dawley rats. The present invention also provides evidence on the safety of selective doses of TQRF, TQ, NSO and NSP by measuring the toxicity parameters including Alanine aminotransferase (ALT), Gamma-glutamyltranspeptidase (GGT), urea and creatinine of plasma collected from experimental rats and rabbits.

The in vivo study using rat shows that treatment of rats with TQRF and TQ at different doses for 8 weeks caused significant decrease in the plasma total cholesterol (TC) and low density lipoprotein cholesterol (LDLC) compared to controlled group of rats. The method includes administrating both TQRF and TQ in emulsion form orally at doses ranged between 0.5-1.5 g/kg body weight of TQRF and 20-100 mg/kg body weight of TQ using Sprague-Dawley rats fed with diets supplemented with 1% cholesterol for 8 weeks.

Similarly, the experimental hypercholesterolemia induced rabbits that were treated with NSP and NSO shows a significant reduction of TC and LDLC which were observed at weeks 2, 4, 6 and 8 of treatments compared to the controlled rabbits. Treatment of rabbits with NSP and NSO showed a significant increase in plasma HDL levels at weeks 4, 6 and 8 of treatment. The activity of NSP and NSO in treating hypercholesterolemia rabbits were similar to the simvastatin (ST) treatment.

The invention also provides in vivo antioxidant activity of nutraceuticals prepared from Nigella sativa. There was a significant increase of total antioxidant status (TAS) in plasma collected from NSP and NSO treated rabbits after 8 weeks of treatment compared to untreated rabbits. The analysis of .OH activity of TQRF and TQ treated rats were carried out using ESR. The findings demonstrate that TQ and TQRF are effective antioxidants with a scavenging rate for .OH. Treating rabbits with NSP and NSO showed strong inhibition of plaque formation in the aorta with significant decrease in the tunica intima to tunica media ratio compared to the control rabbits.

To understand the molecular basis of the hypocholesterolemic effect of TQRF and TQ at different doses, mRNA expression levels of selected genes including LDLR, HMG-COAR, beta actin, Apo A-1, Apo E and Apo B100 of human HepG2 cells and liver tissues isolated from experimental rats were analyzed by quantitative real time PCR. In this invention, in vitro and in vivo study using rats showed that TQRF and TQ treatments could regulate the key genes involved in cholesterol metabolism, LDLR, Apo E and Apo A-I were up-regulated, whereas the HMG-COAR and Apo B100 were down-regulated compared to the control. The regulation of these genes was at transcription level suggesting that TQRF and TQ regulated cholesterol through different events, including cholesterol synthesis, and cholesterol uptake.

The non toxic property of NSP, NSO, TQRF and TQ in animals is shown by key hepatic and kidney enzyme stability and organ integrity and safe for the selective doses used. Our results support the traditional uses of Nigella sativa, in which the seed and its oil have been used safely for thousands of years in the Middle East and South Asia as a natural treatment for treating many illnesses.

### EXAMPLES

The following examples are for purposes of illustration only, and are not intended to limit the scope of the appended claims.

### Example 1

### In Vivo Hypocholesterolemic Effect of TQRF and TQ (Rat Study)

### 1.1 Animal Maintenance and Diets

Ninety male Sprague-Dawley rats weighing 150-200 g were used. The experiment carried out was according to the guidelines for the care and use of animals approved by Animal Care and Use Committee (ACUC) of Faculty of Medicine and Health Sciences, Universiti Putra Malaysia. They were fed initially standard rat chow pellets for two weeks for adaptation.

### 1.2 Diet Preparation

Diet was prepared in the Laboratory of Molecular Biomedicine Institute of Bioscience Universiti Putra Malaysia. Normal rat chow was ground using an electric grinder (Manesty 3001 UK), weighed, mixed with starch (5% of the diet), cholesterol (1% of the diet), 200 ml water tap and placed on a dish covered with aluminum foil. The diet was pelleted, dried in an oven at 45-50° C. overnight and kept at 4° C. About 20 g of pellet was given to each rat daily.

### 1.3 Treatments Preparation

Both TQRF and TQ were administrated to the rats orally in emulsion form. Calculated amount of TQRF (6 g) was mixed well with tween 80 (0.5 g), 20 ml distilled water. The mixture was homogenized at 13000 rpm for 3-5 minutes. Rats were fed 2 ml of the emulsion freshly prepared daily. The TQ emulsion was prepared by dissolving calculated amount of TQ (160 mg) in 1 ml of triolein and prepared as TQRF emulsion. Triolien emulsion was prepared by mixing 1 ml of triolein with 20 ml water and homogenized at 13000 rpm for 2-3 min.

### 1.4 Rats Grouped

Nine experimental rat groups were established (10 rats per each group). The groups were as follows. Group 1: negative control animals (NC) were fed a standard commercial diet. Group 2: positive cholesterol group (PC), were fed commercial diet supplemented with 1% (w:w) cholesterol. Groups 3-5 were fed commercial diet supplemented with 1% cholesterol and TQRF emulsion, TQRF 1=group that treated with TQRF at 0.5 g/kg, TQRF 2=group that treated with TQRF at dose 1 g/kg, TQRF 3=group that treated with TQRF at dose of 1.5 g/kg. Groups 6-8: were fed commercial diet supplemented with 1% cholesterol and TQ emulsion, TQ 1=group that treated with TQ at dose 20 mg/kg for 8 weeks, TQ 2=group that treated with TQ at dose of 50 mg/kg for 8 weeks, TQ 3=group that treated with TQ at dose 20 mg/kg

### 1.5 Results

Total Cholesterol Level (TC)

TABLE 1
TC levels in Experimental Rat
TC levels (mmol/l) in experimental rats

| | Blood collection time | | |
|---|---|---|---|
| | | After 4 | After 8 |
| Group | Baseline | weeks | weeks |
| NC | 0.88 ± 0.28 | 0.97 ± 0.20 ^{b} | 0.98 ± 0.16 ^{a} |
| PC | 0.94 ± 0.27 | 1.75 ± 0.34 ^{a} | 2.13 ± 0.33 ^{b} |
| TQRF 1 | 0.88 ± 0.45 | 1.74 ± 0.23 ^{a} | 1.19 ± 0.24 ^{a} |
| TQRF 2 | 0.91 ± 0.25 | 1.30 ± 0.18 ^{b} | 1.16 ± 0.28 ^{a} |
| TQRF 3 | 0.87 ± 0.26 | 1.24 ± 0.12 ^{b} | 0.95 ± 0.19 ^{a} |
| TRiolein | 0.80 ± 0.12 | 1.66 ± 0.16 ^{c, a} | 1.74 ± 0.46 ^{c} |
| TQ1 | 0.85 ± 0.19 | 1.49 ± 0.24 ^{b} | 1.48 ± 0.22 ^{a, c} |
| TQ2 | 0.97 ± 0.05 | 1.47 ± 0.31 ^{b} | 1.01 ± 0.34 ^{a} |
| TQ3 | 0.87 ± 0.26 | 1.41 ± 0.48 ^{b} | 0.94 ± 0.30 ^{a} |

Results are expressed as means±SDV of 7 rats per group. PC=cholesterol positive control, NC=negative control, TQRF 1=group that treated with TQRF at 0.5 g/kg, TQRF 2=group that treated with TQRF at dose 1 g/kg, TQRF 3=group that treated with TQRF at dose of 1.5 g/kg. TQ 1=group that treated with TQ at dose 20 mg/kg for 8 weeks, TQ 2=group that treated with TQ at dose of 50 mg/kg for 8 weeks, TQ 3=group that treated with TQ at dose 20 mg/kg for 8 weeks.

In this study, TQRF and TQ treatments lowered plasma cholesterol levels in rats fed with cholesterol diet. Analysis of lipoprotein distribution showed that the reduction of cholesterol could be attributed to changes in level of LDL cholesterol. TQ and TQRF produced a dose-dependent reduction of plasma cholesterol with higher doses begin more effective.

### Example 2

### Hypocholesterolemic Effect of NSP and NSO in Hypercholesterolemia Induced Rabbits

### 2.1 Experimental Procedures

Twenty five male New Zealand white rabbits weighing 1.25-2.5 kg were used for this study. Rabbits were randomly divided into negative control (NC), (n=5), which was fed a normal diet and hypercholesterolemia induced rabbits (n=20) which were fed normal diet with 0.5% cholesterol for three weeks. Hypercholesterolemic rabbits were then divided into 4 subgroups; a group that was fed with a normal diet without any treatment and used as a cholesterol control (PC), a group that was fed a normal diet+3.5 g/kg/day NSP, a group that was fed a normal diet+1.5 g/kg/day NSO and a group that was fed a normal diet+10 mg/kg/day simvastatin from Ranbaxy (Pharmaniaga Logistics Sdn. Bhd. 260790-T) by force feeding.

### 2.2 Results

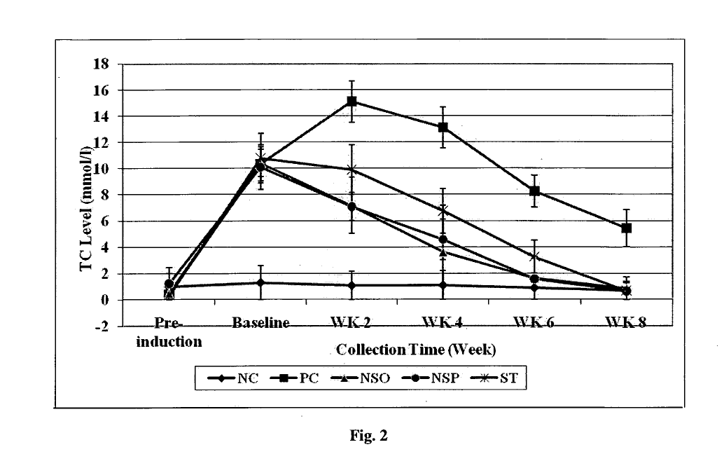

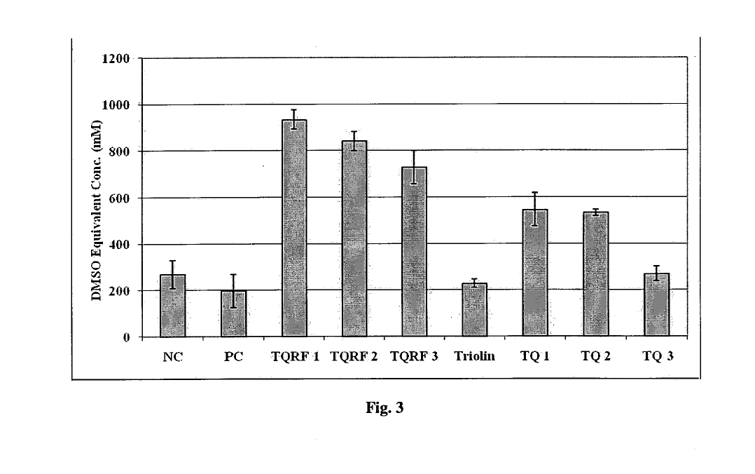

There was a significant reduction of plasma TC levels in NSP (52%) and NSO (53%) groups as compared to the PC group after 2 weeks of treatment when compared to the PC (see FIG. 2). Analysis of lipoprotein distribution showed significant reduction of TC and LDL levels and increased HDL levels.

### Example 3

### Antioxidant Activity of TQRF and TQ In Vivo

Hydroxyl Radical Scavenging Activity of Rat Plasma Treated with Different Doses of TQRF and TQ using ESR Spectrometer Electron Spin Resonance (ESR) Techniques

### 3.1 Experimental Procedures

Hydroxyl radical scavenging (OH.) activity of plasma collected from the experiment was detected using ESR, the measurements were made as follows: magnetic field: 336.450±5 mT; power: 8 Mw, modulation frequency: 100 KHz; modulation width 0.1 Mt amplitude: 1×0.1 roT; response time: 0.1 seconds; amplitude: 50; and the sweep time: 2 minutes. ESR spectra were measured at room temperature, 25° C. Data analysis was performed using a computerized program (version 5.2 for JES-FR 30) connected to the Free Radical Monitor.

### 3.2 Results

### Hydroxyl Radical Scavenging Activity of TQ and TQRF

There were significant differences in the hydroxyl radical scavenging in treated rats with different doses of TQ and TQRF compared to the control group (see FIG. 3). Previous studies showed that feeding rats with cholesterol diet increased the OH. production. Our results demonstrated that treatment with both TQRF and TQ are able to directly scavenge .OH and protected LDL from oxidation, and reduce serum cholesterol levels.

### Example 4

### Total Antioxidant Activity of NSP and NSO in Hypercholesterolemia Induced Rabbits

### 4.1 Experimental Procedures

The total antioxidant status (TAS) of plasma collected from experimental rabbits was assayed using Selectra E with commercial kits (Randox, Crumlin, Co. Antrim, UK). A significant increase of TAS activity was observed in all the treatment groups (NSP, NSO as compared to the PC group at week 8 of the treatment.

### 4.2 Results

In this invention, Nigella sativa treatment, either in powder or as oil form showed significant increases in plasma TAS activity as compared to control rabbits. This could explain the antioxidant effects of Nigella sativa and its oil extracted.

TABLE 2
Total antioxidant status (TAS) of plasma
in hypercholesterolemia induced rabbits

Total antioxidant status (mmol/l)

| Group | (baseline) | Week 8 of treatment |
|---|---|---|
| NC | 1.58 ± 0.03^{a} | 1.57 ± 0.03 |
| PC | 1.52 ± 0.03^{b} | 1.46 ± 0.03^{a} |
| NSP | 1.54 ± 0.03^{b} | 1.70 ± 0.03^{b} |
| NSO | 1.52 ± 0.03^{b} | 1.72 ± 0.04^{b} |

Results are expressed as means±SD of five animals per group. Within a column, values with the same superscript letters are not significant different from each other at p<0.05. PC=positive control, NC=negative control, NSO=Nigella sativa seed oil and NSP=Nigella sativa seeds in powder form. Comparison of plasma TAS (mmol/l) values at baseline of the experiment and the end of the treatment.

### Example 5

### Antiatherogenic Activity of NSP and NSO in Hypercholesterolemia Induced Rabbits

### 5.1 Experimental Procedures

At the end of the experiment, all rabbits were dissected and the aortas were removed. For each aorta, the plaque formatic was analyzed for the average determination of the thickness of the intima, media and the ratio of intima: media for 3 rabbits per each group was detected using an image-analysis system (OLYMPUS BX41) interfaced to a Zeiss Axioscop microscope.

### 5.2 Results

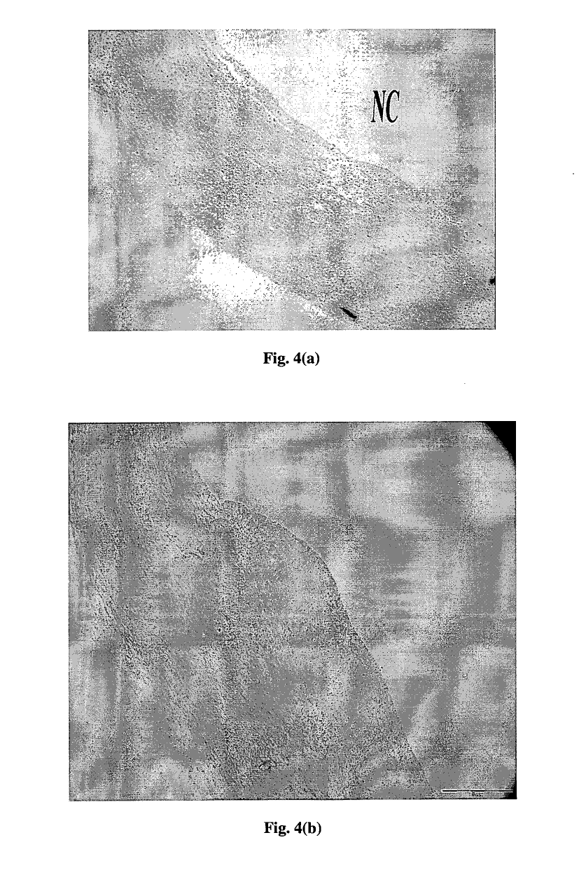

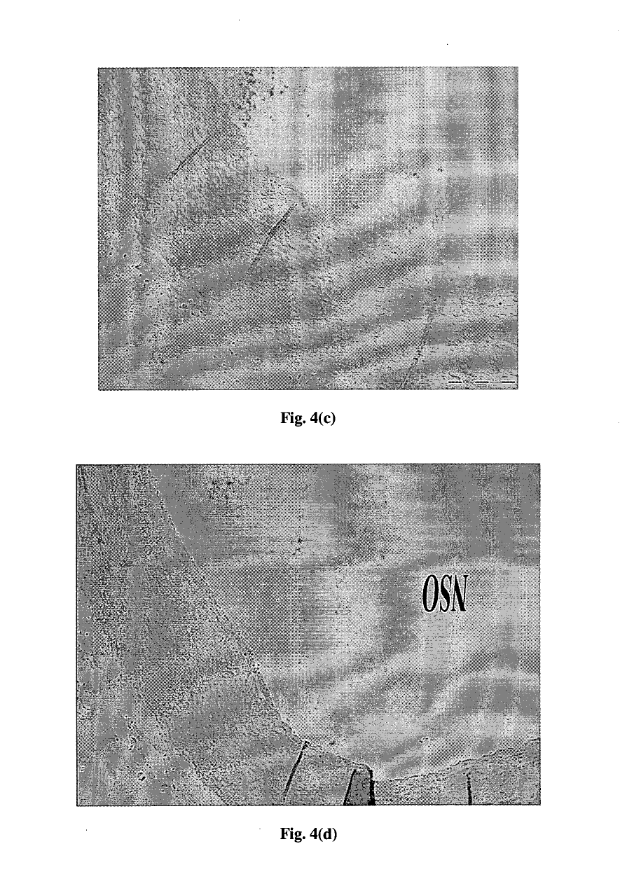

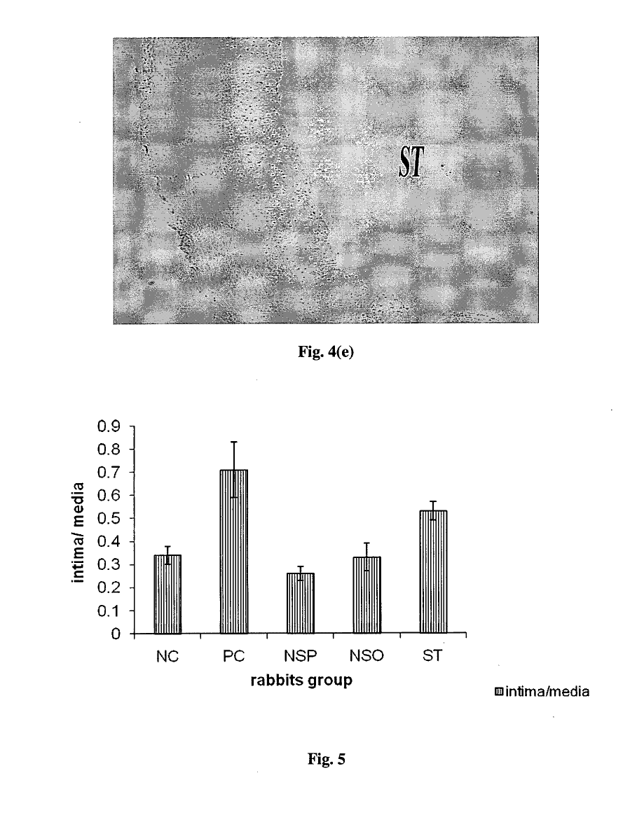

The intima to media ratio was increased significantly in the PC group compared to NSP and NSO groups. The results obtained showed a significant increase between the PC group (71%) and treatment groups (26, 33 and 53% in NSP, NSO and ST groups respectively). Among the treatment groups, NSP and NSO showed significant decrease (p<0.05) in intima:media ratio compared to ST group. Furthermore, among the Nigella sativa groups there was no significant different in intima:media ratio between NSO and NSP group (see FIG. 4, A-F).

### Example 6

### In Vitro and In Vivo Gene Expression Study

This invention provided clear understanding of the molecular basis of the hypocholesterolemic effect of TQRF TQ. The mRNA levels of key genes that involved in cholesterol metabolism including LDLR, HMG-COAR, Apo A-1, Apo E and Apo B100 for HepG2 cells and liver tissues from experimental rats were analyzed using real time PCR. The major effect of TQRF and TQ were concentration-dependent increase on LDLR and Apo E mRNA and suppressed the HMG-COAR and Apo B100 mRNA in treated rats compared to control rats.

### 6.1 Materials and Reagents

The human hepatoblastoma cell line, HepG2 was given from PROF Ciew lab, Faculty of Medicine, UPM. Methanol, Dimethyl sulfoxide (DMSO), 2-propanol of HPLC grade (Fisher Scientific, USA). Membrane filter (0.2 μm), TQ standard, Dulbecco's minimum essential medium (DMEM), fetal bovine serum (FBS), penicillin and streptomycin, trypsin, 25OH, human lipoprotein defiant serum (HLPDS) (Sigma-Aldrich Co., USA). Isopropanol, methanol HPLC grade (BDH Chemicals, UK), MIT (3-(4,5-dimethylthiazole-2-yl)-2,5-diphenyl tetrazolium bromide), sodium bicarbonate, sodium hydroxide (NaOH), HCL, phosphate buffer saline (PBS), tissue culture flasks, 96 well plates, filter unit for media 500 ml, centrifuge tube and sterile tips (Sigma-Aldrich Co., USA). Leicestershire. A TACS™ Annexin V-FITC Apoptosis detection kit for flow cytometry (Promega, USA) RiboPure RNA isolation kit, MMLV (Moloney Murine Leukemia Virus) (Ambion, USA). Quantict Probe Real time PCR master mix (Qiagen INC., AUSA). The oligo (dT) primer and probes supplied by Sigma-Aldrich and Integrated DNA Technologies (IDT, USA)

### 6.2 Experimental Procedures

### 6.2.1 HepG2 Cells Culture

For the determination of mRNA expression levels of LDLR, HMG-COAR, beta actin, Apo A-1, Apo E and Apo B100 by real time PCR, HepG2 cells were plated in 6-well plates at a density of 1.8×10⁵ cells/well for 24 h. The cells were incubated with 10% HLPDS for 24 h with or without adding 2 μg/ml 25OH for another 24 h. The cells were treated with TQRF at two different doses, 80 and 50 μg/ml for 24 h, and with TQ at dose of 2 μg/ml

### 6.2.2 Liver Tissues

At the end of the experiment, all the rats were dissected and liver tissues for RNA isolation were removed, snap frozen with liquid nitrogen within approximately 2 min of death, and stored at −80° C. for gene expression study.

6.2.3 RNA Isolation and cDNA Synthesis

Total RNA was isolated from HepG2 cells and rats liver tissues using RiboPure RNA Isolation Kit according to the manufacturer's instructions. First-strand cDNA was synthesized using MMLV with random primer according to the manufacturer's instructions.

### 6.2.4 Quantitative Real-Time PCR

Real-time quantitative PCR was performed using the Quantict Probe Real time PCR master mix according to the manufacturer's instructions. TaqMan Primers and probes specific for LDLR and HMG-COA genes were designed using the sequence entries from GenBank (Table. 1) Database and synthesis by Integrated DNA Technologies (IDT). Human beta-actin mRNA was used as housekeeping gene, TaqMan Primers and probes specific for human beta-actin gene was synthesis by Sigma Aldrich. The probes were labeled with FAM 3′ end and the fluorophores 6FAM at 5′ end. The real-time quantitative PCR reaction for each sample was carried out in triplicate and each experiment was repeated twice. Briefly, a reaction volume of 25 μl contained 12.5 μl Quantict Probe Real time PCR master mix and 2 μl of 400 nM from each forward and reverse primers, 1 μl of 200 nM from the probe and 1 μl of the template cDNA at concentration of 100 ng and the volume was up to 25 μl by molecular grade water. Real-time PCR amplification of cDNA was carried out for 40 cycles. After an initial incubation for 15 min at 95° C. PCR cycle comprised denaturation for 15 second at 94° C., annealing for 60 second at 60° C. Amplicon size and reaction specificity were confirmed by 2% agarose gel electrophoresis. Analysis of gene expression data was carried out by ΔΔC_{T} method of relative quantification, according to Kenneth, et al, (2001) (32). RotorGene analysis software (version 6.0) was used to analyze all of the real time PCR results.

### 6.3 Results

### Example for Gene Expression Study In Vivo and In Vitro

### 6.3.1 The Effects of TQRF and TQ on the Expression Level of LDLR In Vitro

The mRNA expression level of LDLR gene was increased by three and seven folds in TQRF 50 and TQRF 80 groups respectively compared to control cells. Whereas, mRNA level of LDLR gene was increased by two folds in TQ 2 group compared to control cells (FIG. 5).

### 6.3.2 Regulation of LDLR Gene by TQRF and TQ Treatments In Vivo

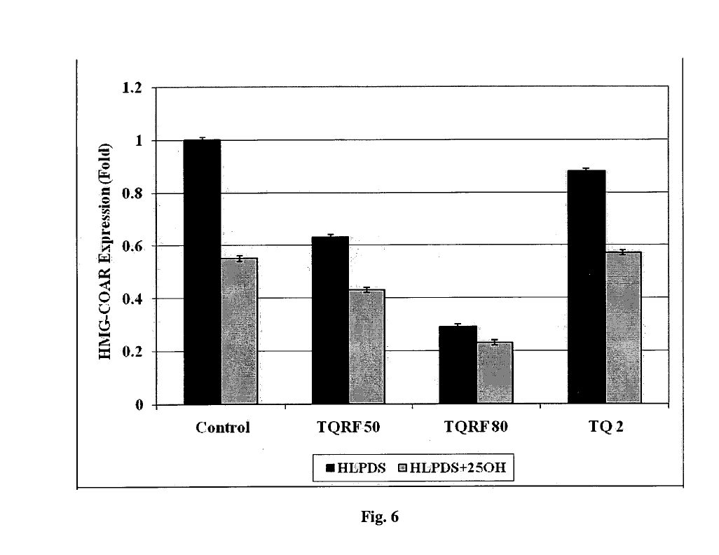

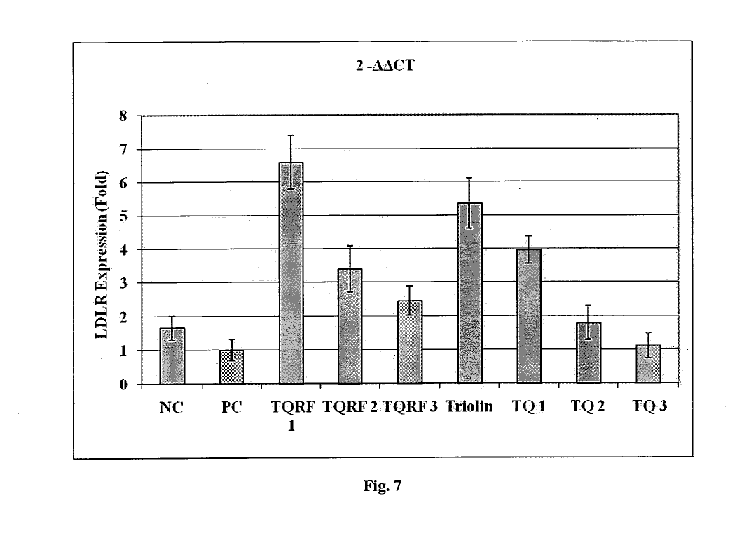

The LDLR mRNA level was up-regulated by three folds in TQRF treated rats at dose of 0.5 g/kg body weight for 8 weeks compared to the untreated rats, whereas, the expression level of LDLR mRNA was six and eight folds in TQRF treated rats at dose of 1 and 1.5 g/kg body weight respectively. Treated rats with 20, 50 and 100 mg/kg body weight of TQ resulted in increased LDLR mRNA levels by two, five and seven folds respectively when compared with untreated rats. The LDLR expression level was increased by increase the dose of both TQRF and TQ (FIG. 6).

### Example 7

### Example for Toxicity Study

### 7.1 Alanine Aminotransferase (ALT)

Toxicity parameter including, ALT of plasma samples collected from experimental rats at baseline, middle of the treatment and the end f the treatment s were measured using analytical kit by kinetic UV assay using Roche Selectra E machine.

### Results

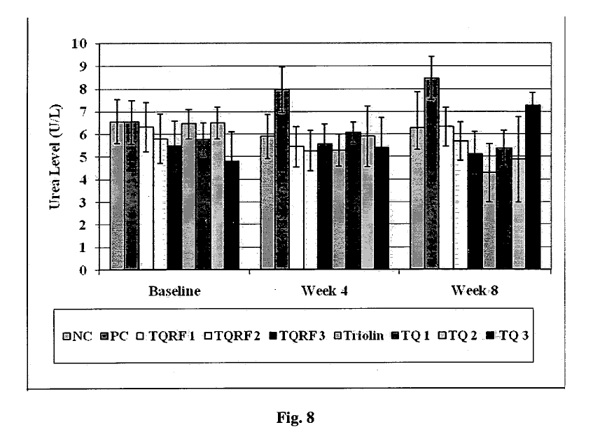

No significant differences were detected in ALT level in all the groups compared to the reference groups (NC) as shown in FIG. 8).

1. A use of an effective amount of thymoquinone rich fraction (TQRF) obtained from Nigella sativa using super critical fluid extraction (SFE) in the manufacture of a medicament for preventing and treating cardiovascular related diseases in a patient in need thereof.

2. The use according to claim 1, wherein the effective amount is between 50 and 80 μg/ml of TQRF.

3. The use of claim 1, wherein TQRF is administered orally or intravenously.

4. The use of claim 1, wherein TQRF is obtained from Nigella sativa seed.

5. The use of claim 1, wherein TQRF contains hypocholestrolemic, anti-oxidant and atherogenic properties for prevention and treatment of cardiovascular related diseases.

6. The use according to claim 1, wherein the patient includes mammal.

7. A use of an effective amount of Nigella sativa powder (NSP) obtained from Nigella sativa using super critical fluid extraction (SFE) in the manufacture of a medicament for preventing and treating cardiovascular related diseases in a patient in need thereof.

8. The use according to claim 7, wherein the effective amount is between 0.5 and 3.5 g/kg of NSP.

9. The use of claim 7, wherein NSP is administered orally or intravenously.

10. The use of claim 7, wherein NSP is obtained from Nigella sativa seed.

11. The use of claim 7, wherein NSP contains hypocholestrolemic, anti-oxidant and atherogenic properties for prevention and treatment of cardiovascular related diseases.

12. The use according to claim 7, wherein the patient includes mammal.

13. A use of an effective amount of Nigella sativa oil (NSO) obtained from Nigella sativa using super critical fluid extraction (SFE) in the manufacture of a medicament for preventing and treating cardiovascular related diseases in a patient in need thereof.

14. The use according to claim 13, wherein the effective amount is between 0.5 and 1.5 g/kg of NSO.

15. The use of claim 13, wherein NSO is administered orally or intravenously.

16. The use of claim 13, wherein NSO is obtained from Nigella sativa seed.

17. The use of claim 13, wherein NSO contains hypocholestrolemic, anti-oxidant and atherogenic properties for prevention and treatment of cardiovascular related diseases.

18. The use according to claim 13, wherein the patient includes mammal.

19. A use of an effective amount of Thymoquinone (TQ) emulsion extracted from Nigella sativa oil (NSO) obtained from Nigella sativa using super critical fluid extraction (SFE) in the manufacture of a medicament for preventing and treating cardiovascular related diseases in a patient in need thereof.

20. The use according to claim 19, wherein the effective amount is between 1 and 100 mg/kg body weight of TQ.

21. The use of claim 19, wherein TO emulsion is administered orally or intravenously.

22. The use of claim 19, wherein TQ emulsion is obtained from Nigella sativa seed oil.

23. The use of claim 19, wherein TQ emulsion contains hypocholestrolemic, anti-oxidant and atherogenic properties for prevention and treatment of cardiovascular related diseases.

24. The use according to claim 19, wherein the patient includes mammal.
